(19) United States
(12) Patent Application Publication
(10) Pub. No.: US 2021/0204908 A1
(43) Pub. Date: Jul. 8, 2021

(54) METHOD AND SYSTEM FOR ASSISTED ULTRASOUND SCAN PLANE IDENTIFICATION BASED ON M-MODE ANALYSIS

(71) Applicant: GE Precision Healthcare LLC, Milwaukee, WI (US)

(72) Inventor: Christian Fritz Perrey, Mondsee (AT)

(21) Appl. No.: 16/737,599

(22) Filed: Jan. 8, 2020

(51) Int. Cl. A61B 8/14; A61B 8/00

(52) U.S. Cl. CPC A61B 8/145; A61B 8/463; A61B 8/465

(57) ABSTRACT

A system and method for assisting the identification of standard ultrasound scan planes based on M-mode analysis is provided. The method includes receiving and displaying an ultrasound scan plane of an anatomy. The method includes positioning one or more M-mode lines in the ultrasound scan plane. The method includes providing an M-mode trace for each of the one or more M-mode lines. The method includes providing feedback based on the M-mode trace for each of the one or more M-mode lines with reference to one or more characteristic trace patterns of a standard view. The method includes receiving and displaying an updated ultrasound scan plane of the anatomy manipulated in response to the feedback.

### FIELD

Certain embodiments relate to ultrasound imaging. More specifically, certain embodiments relate to a method and system for assisting the identification of standard ultrasound scan planes based on M-mode analysis. The system may provide one or more M-mode traces corresponding to one or more M-mode lines positioned in a received ultrasound scan plane. The system may provide feedback regarding the one or more M-mode traces with reference to characteristic trace patterns corresponding to standard ultrasound scan planes.

### BACKGROUND

Ultrasound imaging is a medical imaging technique for imaging organs and soft tissues in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a valuable, non-invasive tool for diagnosing various medical conditions. Acquired ultrasound data may be analyzed and/or processed to visualize anatomical structures evaluated by a medical professional to perform the diagnosis. Typical ultrasound examinations are performed by acquiring a series of ultrasound images in different planes. In some cases, less experienced ultrasound operators may have difficulty acquiring several of the image views for performing a diagnosis. For example, a fetal heart ultrasound examination may involve acquiring various views of the fetal heart, such as one or more of a four chamber (4CH) view, a three vessel and trachea (3VT) view, left ventricular and right ventricular outflow tract views, short axis views, a long axis view, an aortic arch view, a ductal arch view, superior and inferior vena cava views, and/or any suitable fetal heart view. Difficulties obtaining one or more desired views may result in abnormalities going undetected.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for assisting the identification of standard ultrasound scan planes based on M-mode analysis, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to assist the identification of standard ultrasound scan planes based on M-mode analysis, in accordance with various embodiments.

FIG. 2 is an exemplary display of an ultrasound scan plane having an M-mode line and a corresponding M-mode trace, in accordance with various embodiments.

FIG. 3 is an exemplary display of an ultrasound scan plane having M-mode lines and corresponding M-mode traces, in accordance with various embodiments.

FIG. 4 is a flow chart illustrating exemplary steps that may be utilized for assisting the identification of standard ultrasound scan planes based on M-mode analysis, in accordance with exemplary embodiments.

### DETAILED DESCRIPTION

Certain embodiments may be found in a method and system for assisting the identification of standard ultrasound scan planes based on M-mode analysis. The system may provide one or more M-mode traces corresponding to one or more M-mode lines positioned in a received ultrasound scan plane. The system may provide feedback regarding the one or more M-mode traces with reference to characteristic trace patterns corresponding to standard ultrasound scan planes. Various embodiments have the technical effect of identifying standard ultrasound scan planes based on M-mode analysis of received ultrasound scan planes with reference to characteristic trace patterns of standard ultrasound scan planes.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams. Also, as used herein, a 3D image or 3D volume includes a volumetric sequence (also known as four-dimensional (4D) imaging).

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

Although the exemplary embodiments described below are presented with respect to the acquisition of ultrasound images of a fetus, the disclosure is not limited to fetal ultrasound examinations. Instead, aspects of the present disclosure are applicable to ultrasound examinations of any suitable anatomical structure for diagnosing any suitable condition.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

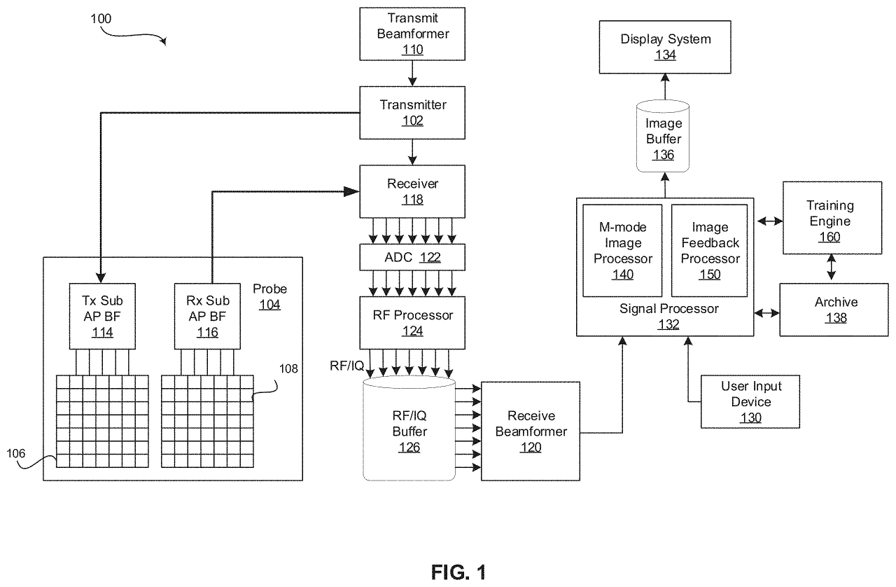

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to assist the identification of standard ultrasound scan planes based on M-mode analysis, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, an archive 138, and a training engine 160.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as a fetus, a spine, an endometrium of a uterus, a heart, a blood vessel, or any suitable anatomical structure. In various embodiments, the ultrasound probe 104 may be operable to acquire ultrasound scan planes at different rotational and/or tilt angles without physically moving the ultrasound probe. In an exemplary embodiment, the ultrasound probe 104 may include a one dimensional transducer array that can be mechanically oriented in a plurality of orientations by a motor in response to instructions from the signal processor 132. In another embodiment, the probe 104 includes a 2D array of ultrasound elements operable to electronically transmit ultrasonic signals and acquire ultrasound data in any orientation in three-dimensional space, called a four dimensional (e4D) matrix probe. For example, the e4D ultrasound probe 104 may be the GE 4Vc-D four-dimensional (4D) matrix cardiac probe. The processing of the acquired images in any steered direction can be performed partially or completely by probe-internal sub-aperture processing, by system-side software beamforming, or by beamforming in hardware.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, position M-mode lines, manipulate ultrasound volumes, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, the archive 138, and/or the training engine 160. The user input device 130 may include button(s), rotary encoder(s), a touchscreen, a touch pad, a trackball, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

In various embodiments, the user input device 130 may be operable to select an examination type having an associated protocol defining image views for acquisition or retrieval. For example, a user may select a fetal heart ultrasound examination or any suitable examination type. The selected examination type may be associated with a number of defined image views for acquisition or retrieval. For example, in the case of a fetal heart ultrasound examination, the examination may be associated with a four chamber (4CH) view, a three vessel and trachea (3VT) view, left ventricular and right ventricular outflow tract views, short axis views (low for ventricles and high for outflow tracts), a long axis view, an aortic arch view, a ductal arch view, superior and inferior vena cava views, and/or any suitable views. In an exemplary embodiment, the user input device 130 may be operable to position one or more M-mode lines in received ultrasound scan planes. For example, in the case of a fetal heart ultrasound examination involving the acquisition or retrieval of a 4CH view, the user input device 130 may be used to provide one or more M-mode lines at locations and orientations for performing measurements, such as a mitral annular plane systolic excursion (MAPSE) measurement, a tricuspid annular plane systolic excursion (TAPSE) measurement, or any suitable measurement. In certain embodiment, the user input device 130 may be operable to manipulate ultrasound image volumes to selected ultrasound scan planes. For example, the user input device 130 may include various rotary encoders, buttons, touchscreen, and the like for rotating, tilting, and/or otherwise navigating the image volume to a desired scan plane.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an M-mode image processor 140 and an image feedback processor 150. The signal processor 132 may be capable of receiving input information from a user input device 130 and/or archive 138, generating an output displayable by a display system 134, and manipulating the output in response to input information from a user input device 130, among other things. The signal processor 132, including the M-mode image processor 140 and the image feedback processor 150, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an M-mode image processor 140 that comprises suitable logic, circuitry, interfaces and/or code that may be operable to position one or more M-mode lines and provide an M-mode trace for each of the positioned M-mode lines. For example, the M-mode image processor 140 may position one or more M-mode lines in a received ultrasound scan plane automatically or in response to an instruction received via the user input device 130. As an example, an operator may acquire or retrieve an ultrasound image of an anatomical structure, such as a fetal heart or any suitable anatomical structure. In various embodiments, the M-mode image processor 140 may be configured to manually position the M-mode line(s). For example, the operator may provide a user instruction via the user input device 130 to the M-mode image processor 140 to select a location and orientation of an M-mode line on a received ultrasound scan plane.

Additionally and/or alternatively, the M-mode image processor 140 may comprise suitable logic, circuitry, interfaces and/or code that may be operable to automatically position the M-mode line(s) in the received ultrasound scan plane. For example, the M-mode image processor 140 may position the M-mode line(s) at pre-defined location(s) and orientation(s) on the ultrasound scan plane. The pre-defined location(s) and orientation(s) may correspond with a measurement type, such as a MAPSE measurement, TAPSE measurement, or any suitable measurement. As another example, the M-mode image processor 140 may position the M-mode line(s) based on automated analysis of the received ultrasound scan plane. For example, the M-mode image processor 140 may include artificial intelligence image analysis algorithms, one or more deep neural networks (e.g., a convolutional neural network) and/or may utilize any suitable form of artificial intelligence image analysis techniques or machine learning processing functionality configured to analyze received ultrasound image planes to identify a position (i.e., location and orientation) to place the M-mode line(s). As an example, the M-mode image processor 140 may be provided as one or more deep neural networks that may be made up of, for example, an input layer, an output layer, and one or more hidden layers in between the input and output layers. Each of the layers may be made up of a plurality of processing nodes that may be referred to as neurons. For example, the M-mode image processor 140 may include an input layer having a neuron for each pixel or a group of pixels from a scan plane of an anatomical structure. The output layer may have a neuron corresponding to a plurality of M-mode line positions. As an example, if performing an ultrasound-based fetal heart examination, the output layer of one deep neural network may include neurons for one or more M-mode line position in a 4CH view, a 3VT view, and the like. Other ultrasound procedures may utilize output layers that include neurons for M-mode line positions corresponding with any suitable image view of any suitable anatomical structure. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the M-mode image processor 140 deep neural network (e.g., convolutional neural network) may position M-mode line(s) in ultrasound image scan planes with a high degree of accuracy. The automated M-mode line placement by the M-mode image processor 140 may expedite the identification of a desired ultrasound scan plane by minimizing the amount of probe movement to acquire, or volume manipulation to obtain, the desired scan plane.

The M-mode image processor 140 may comprise suitable logic, circuitry, interfaces and/or code that may be operable to generate an M-mode trace corresponding to each of the one or more M-mode lines for presentation at the display system 134. For example, the M-mode image processor 140 may be configured to generate and present one M-mode trace if one M-mode line is placed in the ultrasound scan plane. As another example, the M-mode image processor 140 may be configured to generate and present two M-mode traces if two M-mode lines are placed in the ultrasound scan plane. The ultrasound scan plane having the M-mode line(s) and the corresponding M-mode trace(s) may be provided to the image feedback processor 150 and/or may be stored at archive 138 and/or any suitable data storage medium.

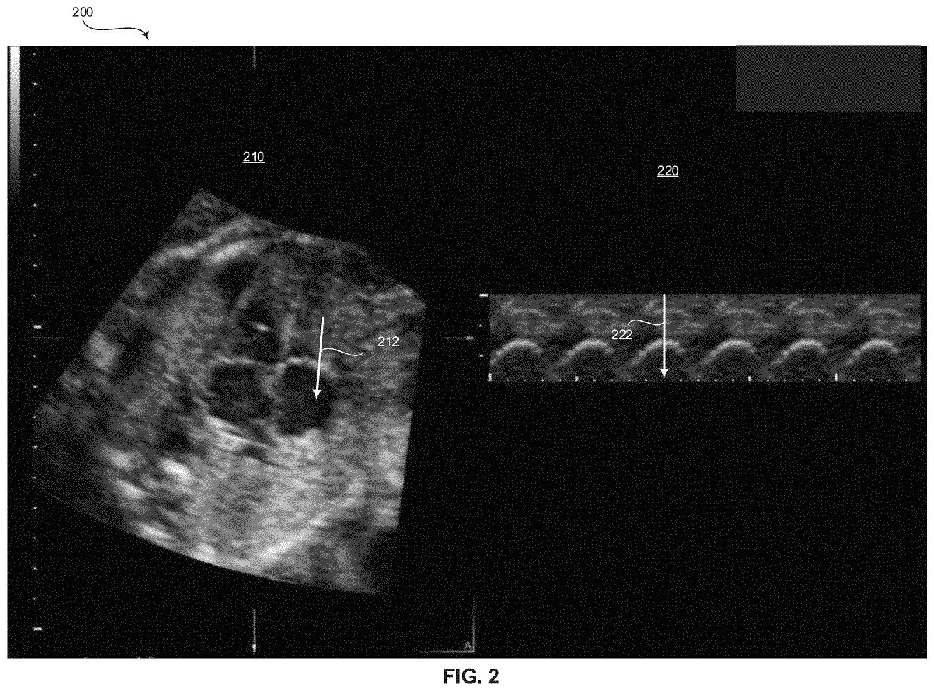

FIG. 2 is an exemplary display 200 of an ultrasound scan plane 210 having an M-mode line 212 and a corresponding M-mode trace 220, in accordance with various embodiments. Referring to FIG. 2, the display 200 may include an ultrasound scan plane 210 and an M-mode trace 220. The ultrasound scan plane 210 may include an M-mode line 212. The M-mode trace 220 may include a trace line 222. The M-mode trace 220 is M-mode image data corresponding with the M-mode line 212 in the ultrasound scan plane 210 over time. The current time is identified by the trace line 222. In various embodiments, the ultrasound scan plane 210 may be acquired by the ultrasound system 100 or retrieved from archive 138 or any suitable data storage medium. The ultrasound scan plane 210 may be displayed 200 at a display system 134 of the ultrasound system 100 or a display system of a workstation. An M-mode image processor 140 of a signal processor 134 of the ultrasound system 100 (or a processor of a workstation) may place an M-mode line 212 in the ultrasound scan plane 210 provided in the display 200. The M-mode line 212 may be placed, for example, in response to a user input, at a pre-defined location and orientation, or at a location and orientation determined by automated analysis of the ultrasound scan plane 210. The M-mode image processor 140 may be configured to generate the M-mode trace 220 corresponding with the M-mode line 212 in the ultrasound scan plane 210. The M-mode trace 220 may be presented at a display 200 of the display system 134 and/or stored at archive 134 or any suitable data storage medium.

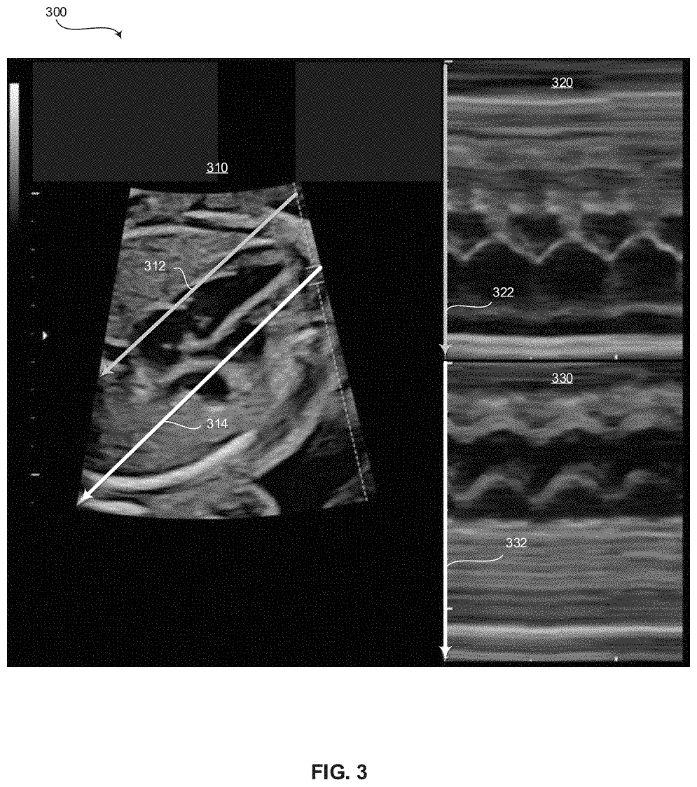

FIG. 3 is an exemplary display 300 of an ultrasound scan plane 310 having M-mode lines 312, 314 and corresponding M-mode traces 320, 330, in accordance with various embodiments. Referring to FIG. 3, the display 300 may include an ultrasound scan plane 310 and M-mode traces 320, 330. The ultrasound scan plane 310 may include M-mode lines 312, 314. The M-mode traces 320, 330 may each include a trace line 322, 332. Each of the M-mode traces 320, 330 is M-mode image data corresponding with one of the M-mode lines 312, 314 of the ultrasound scan plane 310 over time. The current time in each M-mode trace 320, 330 is identified by the trace line 322, 332. In various embodiments, the ultrasound scan plane 310 may be acquired by the ultrasound system 100 or retrieved from archive 138 or any suitable data storage medium. The ultrasound scan plane 310 may be displayed 300 at a display system 134 of the ultrasound system 100 or a display system of a workstation. An M-mode image processor 140 of a signal processor 134 of the ultrasound system 100 (or a processor of a workstation) may place the M-mode lines 312, 314 in the ultrasound scan plane 310 provided in the display 300. The M-mode lines 312, 314 may be placed, for example, in response to user inputs, at pre-defined locations and orientations, or at locations and orientations determined by automated analysis of the ultrasound scan plane 310. The M-mode image processor 140 may be configured to generate each the M-mode traces 320, 330 corresponding with the M-mode lines 312, 314 in the ultrasound scan plane 310. The M-mode traces 320, 330 may be presented at a display 300 of the display system 134 and/or stored at archive 134 or any suitable data storage medium.

Referring again to FIG. 1, the signal processor 132 may include an image feedback processor 150 that comprises suitable logic, circuitry, interfaces and/or code that may be operable to provide feedback regarding whether the presented ultrasound scan plane 210, 310 provides a standard ultrasound scan plane view. In a representative embodiment, the feedback provided by the image feedback processor 150 may include an M-mode trace template overlaid on, or adjacent to, the presented M-mode trace 220, 320, 330 at the display 200, 300 of the display system 134. For example, the image feedback processor 150 may retrieve a reference M-mode trace template (i.e., a characteristic trace pattern) corresponding to a position (i.e., location and orientation) of the M-mode line 212, 312, 314 in the ultrasound scan plane 210, 310 and the desired standard view from the archive 138 and/or any suitable data storage medium. As an example, the image feedback processor 150 may retrieve a reference M-mode trace for a particular position of an M-mode line 212, 312, 314 in a standard 4CH view. The operator may manipulate the ultrasound probe 104 to acquire a 2D or 3D image or manipulate a 3D ultrasound volume to obtain, an ultrasound scan plane 210, 310 having a corresponding M-mode trace 220, 320, 330 that matches reference traces for the 4CH view. The operator may understand that the 4CH view has been obtained when the M-mode trace(s) 220, 320, 330 corresponding with the presented ultrasound scan plane 210, 310 match the reference trace template(s) presented at the display system 134.

In an exemplary embodiment, the image feedback processor 150 may comprise suitable logic, circuitry, interfaces and/or code that may be operable to analyze generated M-mode trace(s) 220, 320, 330 to determine whether a standard view is provided. The image feedback processor 150 may include image detection algorithms, one or more deep neural networks (e.g., a convolutional neural network) and/or may utilize any suitable form of image detection techniques or machine learning processing functionality configured to automatically identify standard views of an anatomical structure provided in the M-mode trace(s) 220, 320, 330. For example, the image feedback processor 150 may be made up of an input layer, an output layer, and one or more hidden layers in between the input and output layers. Each of the layers may be made up of a plurality of processing nodes that may be referred to as neurons. For example, the input layer may have a neuron for each pixel or a group of pixels from an M-mode trace 220, 320, 330 corresponding with an M-mode line 212, 312, 314 in a presented ultrasound scan plane 210, 310. The output layer may have a neuron corresponding to each degree of closeness to a pre-defined standard view. As an example, if imaging a fetal heart, the output layer may include neurons for a 4CH view and various degrees of closeness to the 4CH view or any suitable fetal heart view. Each neuron of each layer may perform a processing function and pass the processed M-mode trace information to one of a plurality of neurons of a downstream layer for further processing. As an example, neurons of a first layer may learn to recognize edges of structure in the M-mode trace data. The neurons of a second layer may learn to recognize shapes based on the detected edges from the first layer. The neurons of a third layer may learn positions of the recognized shapes relative to landmarks in the M-mode trace data. The processing performed by the image feedback processor 150 deep neural network (e.g., convolutional neural network) may identify degrees of closeness to a desired standard view of an anatomical structure in M-mode trace data with a high degree of probability.

In various embodiments, the feedback may include presenting a message at the display system 134 when the ultrasound scan plane 210, 310 matches a standard view based on M-mode analysis. For example, the image feedback processor 150 may provide a view title (e.g., 4CH) at the display 200, 300 of the display system 134 when the presented ultrasound scan plane 210, 310 corresponds with a standard view based on the M-mode analysis. As another example, the image feedback processor 150 may provide other visual cues, audible cues, and/or physical cues when the presented M-mode trace(s) 220, 320, 330 match reference trace(s) of the standard view. Examples of other visual cues may include a symbol (e.g., green light on a traffic symbol when correct view is presented, yellow light when close to correct view, red light when farther away from correct view), a solid or flashing icon (e.g., solid when view is achieved, faster flashing when close, slower flashing when far), textual or numerical messages (e.g., indicating degrees of closeness and/or achieved standard view), and the like. The audible cues may include audible messages, tones, or the like corresponding with the presentation of a standard view and/or a degree of closeness to the standard view. The physical cues may include probe vibration when a standard view has been obtained, among other things.

In certain embodiments, the feedback may include instructions for automatically or manually rotating and/or tilting the acquisition plane or manipulating 3D volume to obtain the desired standard view. For example, the image feedback processor 150 may be configured to provide directional feedback for manually manipulating a probe 104 or a 3D volume to obtain the selected and/or desired standard view, such as movement instructions, rotation instructions, and/or tilt instructions. As another example, the image feedback processor 150 may comprise suitable logic, circuitry, interfaces and/or code that may be operable to automatically rotate and/or tilt the acquisition of the scan plane or the manipulation of an acquired 3D volume to obtain a standard view. The image feedback processor 150 may be configured to provide rotations and/or tilts based on the M-mode analysis until the desired standard view is obtained. For example, in response to an instruction to obtain a 4CH view of a fetal heart and an ultrasound probe 104 is manually positioned at, or a 3D volume is manually manipulated to, the apical window of a patient, the image feedback processor 150 may automatically rotate and/or tilt the scan plane angle based on the M-mode analysis until a scan plane of the desired view is presented. In various embodiments, the image feedback processor 150 may be configured to analyze the M-mode trace(s) 220, 320, 330 corresponding to the presented ultrasound scan plane 210, 310 to determine whether an acceptable standard view was obtained. If an ultrasound scan plane 210, 310 having an acceptable standard view has been acquired, the ultrasound scan plane 210, 310 may be presented at the display system 134. If the acquired scan plane does not provide an acceptable standard view, the image feedback processor 150 may iteratively acquire additional scan planes or manipulate the 3D volume until an acceptable standard view is identified.

The image feedback processor 150 may be operable to provide the feedback (e.g., template(s), visual cues, audio cues, physical cues, automatic manipulations, etc.) to a user via the ultrasound system 100 (e.g., display 134, probe 104, speakers, etc.).

Still referring to FIG. 1, the display system 134 may be any device capable of communicating visual information to a user. For example, a display system 134 may include a liquid crystal display, a light emitting diode display, and/or any suitable display or displays. The display system 134 may include one or more display screens. For example, the ultrasound scan plane 210, 310 may be presented at a first display screen of the display system 134 and the M-mode trace(s) 220, 320, 330 and/or reference traces may be presented at a second display screen of the display system 134. The display system 134 can be operable to display information from the signal processor 132 and/or archive 138, such as ultrasound scan planes 210, 310, M-mode trace(s) 220, 320, 330, reference trace templates, feedback, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound scan planes 210, 310, M-mode trace(s) 220, 320, 330, reference trace templates, feedback, instructions for generating M-mode trace(s) 220, 320, 330, instructions for selecting reference trace templates, instructions for performing M-mode analysis, and/or instructions for providing feedback, among other things.

The training engine 160 may comprise suitable logic, circuitry, interfaces and/or code that may be operable to train the neurons of the deep neural network(s) of the M-mode image processor 140 and/or the image feedback processor 150. For example, the artificial M-mode image processor 140 and/or the image feedback processor 150 may be trained to automatically position M-mode lines 212, 312, 314 and/or automatically identify closeness to standard views. For example, the training engine 160 may train the deep neural networks of the M-mode image processor 140 and/or the image feedback processor 150 using databases(s) of classified ultrasound scan planes and/or M-mode traces of various structures. As an example, the M-mode image processor 140 and/or the image feedback processor 150 may be trained by the training engine 160 with images of particular anatomical structures to train the M-mode image processor 140 and/or the image feedback processor 150 with respect to the characteristics of the particular structure, such as the appearance of structure edges, the appearance of structure shapes based on the edges, the positions of the shapes relative to landmarks in the image data, and the like. In an exemplary embodiment, the structures may include a fetal heart and/or any suitable organ, bone, nerve, vessel, tissue, or the like. The structural information may include information regarding the edges, shapes, and positions of the fetal heart, organs, bones, nerves, vessels, tissue, and/or the like. In various embodiments, the databases of training images may be stored in the archive 138 or any suitable data storage medium. In certain embodiments, the training engine 160 and/or training image databases may be external system(s) communicatively coupled via a wired or wireless connection to the ultrasound system 100.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

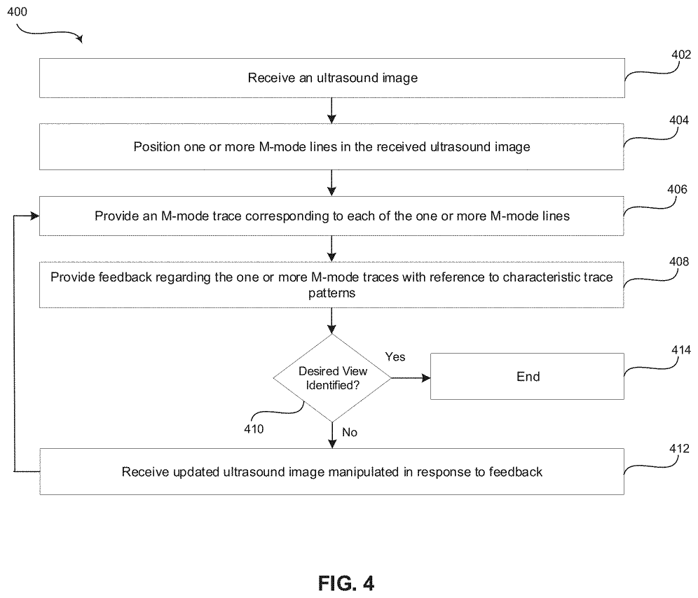

FIG. 4 is a flow chart 400 illustrating exemplary steps 402-414 that may be utilized for assisting the identification of standard ultrasound scan planes based on M-mode analysis, in accordance with exemplary embodiments. Referring to FIG. 4, there is shown a flow chart 400 comprising exemplary steps 402 through 414. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, a signal processor 132 of an ultrasound system 100 or workstation may receive an ultrasound scan plane 210, 310 of an anatomical structure, such as a fetal heart or any suitable anatomical structure. For example, an ultrasound operator may manipulate an ultrasound probe 104 of the ultrasound system 100 to obtain an ultrasound scan plane 210, 310. As another example, an operator may manipulate a 3D volume to obtain an ultrasound scan plane 210, 310. The ultrasound scan plane 210, 310 may be presented by the signal processor 132 at a display system 134.

At step 404, one or more M-mode lines 212, 312, 314 are positioned in the received ultrasound scan plane 210, 310. For example, an M-mode image processor 140 of the signal processor 132 may position one or more M-mode lines 212, 312, 314 in the ultrasound scan plane 210, 310 received at step 602. The M-mode image processor 140 may position the one or more M-mode lines 212, 312, 314 in response to instructions received via a user input device 130, at pre-defined location(s) based on a selected standard view (e.g., 4CH view) and/or measurement type (e.g., MAPSE, TAPSE, or the like), and/or automatically based on automated analysis of the received ultrasound scan plane 210, 310.

At step 406, the signal processor 132 may provide an M-mode trace 220, 320, 330 corresponding to each of the one or more M-mode lines 212, 312, 314 positioned in the ultrasound scan plane 210, 310. For example, the M-mode image processor 140 may generate an M-mode trace 220, 320, 330 for each M-mode line 212, 312, 314 positioned in the ultrasound scan plane 210, 310 at step 404. The M-mode trace(s) 220, 320, 330 may be presented by the M-mode image processor 140 at the display system 134.

At step 408, the signal processor 132 of the ultrasound system 100 or a workstation may provide feedback regarding the one or more M-mode traces 220, 320, 330 with reference to characteristic trace patterns. For example, an image feedback processor 150 of the signal processor 132 may present one or more reference trace templates corresponding with a desired standard view at the display system 134 for comparison with the one or more M-mode traces 220, 320, 330 provided at step 406. As another example, the image feedback processor 150 may analyze the one or more M-mode traces 220, 320, 330 provided at step 406 by applying image detection techniques or machine learning processing functionality configured to automatically identify standard views of an anatomical structure provided in the M-mode trace(s) 220, 320, 330. The image feedback processor 150 may provide visual, audio, and/or physical cues indicating a standard view has been obtained and/or a closeness to a desired standard view. As another example, the image feedback processor 150 may provide instructional feedback for automatically or manually manipulating a probe 104 or an ultrasound volume to achieve the desired standard view.

At step 410, a determination is made regarding whether the desired standard view has been obtained. For example, an operator may manually determine that the ultrasound scan plane 210, 310 matches a characteristic trace pattern provided in a reference trace template. As another example, the feedback provided by the image feedback processor 150 at step 408 may determine that the M-mode trace(s) generated at step 406 correspond with a standard ultrasound scan plane view. If the feedback provided at step 408 indicates the ultrasound scan plane 210, 310 is of the desired or selected standard view, the process 400 ends at step 414. If the feedback provided at step 408 indicates the ultrasound scan plane 210, 310 is not the desired or selected standard view, the process 400 proceeds to step 412.

At step 412, if the feedback provided at step 408 indicates the ultrasound scan plane 210, 310 is not the desired or selected standard view, the signal processor 132 may receive an updated ultrasound scan plane 210, 310 manipulated in response to the feedback. For example, an ultrasound operator may manipulate an ultrasound probe 104 of the ultrasound system 100 to obtain an ultrasound scan plane 210, 310 having a corresponding M-mode trace 220, 320, 330 that substantially matches a characteristic pattern in the reference M-mode trace(s). As another example, an operator may manipulate a 3D volume to obtain an ultrasound scan plane 210, 310 having a corresponding M-mode trace 220, 320, 330 that substantially matches a characteristic pattern in the reference M-mode trace(s). As another example, an operator may manually manipulate the probe 104 or 3D volume based on instructional feedback or degree of closeness feedback provided by the image feedback processor 150. As another example, the image feedback processor 150 may provide instructions applied by the system 100 to automatically obtain an updated scan plane 210, 310. The updated ultrasound scan plane 210, 310 may be presented by the signal processor 132 at a display system 134. The process may then return to step 406 and repeat steps 406 through 412 until the desired and/or selected standard view is obtained.

At step 414, the process 400 ends.

Aspects of the present disclosure provide a method 400 and system 100 for assisting the identification of standard ultrasound scan planes based on M-mode analysis. In accordance with various embodiments, the method 400 may comprise receiving and displaying 402, by at least one processor 132, an ultrasound scan plane 210, 310 of an anatomy. The method 400 may comprise positioning 404, by the at least one processor 132, 140, one or more M-mode lines 212, 312, 314 in the ultrasound scan plane 210, 310. The method 400 may comprise providing 406, by the at least one processor 132, 140, an M-mode trace 220, 320, 330 for each of the one or more M-mode lines 212, 312, 314. The method 400 may comprise providing 408, by the at least one processor 132, 150, feedback based on the M-mode trace 220, 320, 330 for each of the one or more M-mode lines 212, 312, 314 with reference to one or more characteristic trace patterns of a standard view. The method 400 may comprise receiving and displaying 412, by the at least one processor 132, an updated ultrasound scan plane 210, 310 of the anatomy manipulated in response to the feedback.

In an exemplary embodiment, the providing 408 the feedback may comprise selecting and displaying one or more reference M-mode trace templates having the one or more characteristic trace patterns of the standard view. In a representative embodiment, the providing 408 the feedback may comprise analyzing, by the at least one processor 132, 150, the M-mode trace 220, 320, 330 for each of the one or more M-mode lines 212, 312, 314, and providing, by the at least one processor 132, 150, a visual cue, an audio cue, and/or a physical cue indicating whether the standard view has been obtained. In certain embodiments, the providing 408 the feedback may comprise analyzing, by the at least one processor 132, 150, the M-mode trace 220, 320, 330 for each of the one or more M-mode lines 212, 312, 314, and providing, by the at least one processor 132, 150, directional feedback to manually manipulate a three-dimensional (3D) volume or an ultrasound probe to obtain the standard view. In various embodiments, the providing the feedback 408 may comprise analyzing, by the at least one processor 132, 150, the M-mode trace 220, 320, 330 for each of the one or more M-mode lines 212, 312, 314, and providing, by the at least one processor 132, 150, directional feedback to automatically manipulate a three-dimensional (3D) volume or an ultrasound acquisition to obtain the standard view. In an exemplary embodiment, the positioning 404 the one or more M-mode lines 212, 312, 314 in the ultrasound scan plane 210, 310 may comprise analyzing, by the at least one processor 132, 140, the ultrasound scan plane 210, 310 of the anatomy, and automatically positioning, by the at least one processor 132, 140, the one or more M-mode lines 212, 312, 314 based on the analysis. In a representative embodiment, the positioning 404 the one or more M-mode lines 212, 312, 314 in the ultrasound scan plane 210, 310 may comprise positioning, by the at least one processor 132, 140, each of the one or more M-mode lines 212, 312, 314 at a pre-defined location and orientation in the ultrasound scan plane 210, 310. In certain embodiments, the ultrasound scan plane 210, 310 is one of an acquired B-mode image or a selected plane in a retrieved three-dimensional (3D) volume.

Various embodiments provide a system 100 for assisting the identification of standard ultrasound scan planes based on M-mode analysis. The system may comprise at least one processor 132, 140, 150 and a display system 134. The at least one processor 132 may be configured to receive an ultrasound scan plane 210, 310 of an anatomy. The at least one processor 132, 140 may be configured to position one or more M-mode lines 212, 312, 314 in the ultrasound scan plane 210, 310. The at least one processor 132, 140 may be configured to provide an M-mode trace 220, 320, 330 for each of the one or more M-mode lines 212, 312, 314. The at least one processor 132, 150 may be configured to provide feedback based on the M-mode trace 220, 320, 330 for each of the one or more M-mode lines 212, 312, 314 with reference to one or more characteristic trace patterns of a standard view. The at least one processor 132, 150 may be configured to receive an updated ultrasound scan plane 210, 310 of the anatomy manipulated in response to the feedback. The display system 134 may be configured to present the ultrasound scan plane 210, 310, the one or more M-mode lines 212, 312, 314, the M-mode trace 220, 320, 330 for each of the one or more M-mode lines 212, 312, 314, and the updated ultrasound scan plane 210, 310.

In a representative embodiment, the at least one processor 132, 150 may be configured to provide the feedback by selecting and displaying one or more reference M-mode trace templates having the one or more characteristic trace patterns of the standard view. In an exemplary embodiment, the at least one processor 132, 150 may be configured to provide the feedback by analyzing the M-mode trace 220, 320, 330 for each of the one or more M-mode lines 212, 312, 314, and providing a visual cue, an audio cue, and/or a physical cue indicating whether the standard view has been obtained. In a representative embodiment, the at least one processor 132, 150 may be configured to provide the feedback by analyzing the M-mode trace 220, 320, 330 for each of the one or more M-mode lines 212, 312, 314, and providing directional feedback to manually manipulate a three-dimensional (3D) volume or an ultrasound probe 104 to obtain the standard view. In various embodiments, the at least one processor 132, 150 may be configured to provide the feedback by analyzing the M-mode trace 220, 320, 330 for each of the one or more M-mode lines 212, 312, 314, and providing directional feedback to automatically manipulate a three-dimensional (3D) volume or an ultrasound acquisition to obtain the standard view. In certain embodiments, the at least one processor 132, 140 may be configured to position the one or more M-mode lines 212, 312, 314 in the ultrasound scan plane 210, 310 by either analyzing the ultrasound scan plane 210, 310 of the anatomy and automatically positioning the one or more M-mode lines 212, 312, 314 based on the analysis, or positioning each of the one or more M-mode lines 212, 312, 314 at a pre-defined location and orientation in the ultrasound scan plane 210, 310. In an exemplary embodiment, the ultrasound scan plane 210, 310 is one of an acquired B-mode image or a selected plane in a retrieved three-dimensional (3D) volume.

Certain embodiments provide a non-transitory computer readable medium having stored thereon, a computer program having at least one code section. The at least one code section is executable by a machine for causing the machine to perform steps 400. The steps 400 may comprise receiving and displaying 402 an ultrasound scan plane 210, 310 of an anatomy. The steps 400 may comprise positioning 404 one or more M-mode lines 212, 312, 314 in the ultrasound scan plane 210, 310. The steps 400 may comprise providing 406 an M-mode trace 220, 320, 330 for each of the one or more M-mode lines 212, 312, 314. The steps 400 may comprise providing 408 feedback based on the M-mode trace 220, 320, 330 for each of the one or more M-mode lines 212, 312, 314 with reference to one or more characteristic trace patterns of a standard view. The steps 400 may comprise receiving and displaying 412 an updated ultrasound scan plane 210, 310 of the anatomy manipulated in response to the feedback.

In various embodiments, the providing 408 the feedback may comprise selecting and displaying one or more reference M-mode trace templates having the one or more characteristic trace patterns of the standard view. In an exemplary embodiment, the providing 408 the feedback may comprise analyzing the M-mode trace 220, 320, 330 for each of the one or more M-mode lines 212, 312, 314, and one of: providing a visual cue, an audio cue, and/or a physical cue indicating whether the standard view has been obtained; providing directional feedback to manually manipulate a three-dimensional (3D) volume or an ultrasound probe 104 to obtain the standard view; or providing directional feedback to automatically manipulate a three-dimensional (3D) volume or an ultrasound acquisition to obtain the standard view. In a representative embodiment, the positioning 404 the one or more M-mode lines 212, 312, 314 in the ultrasound scan plane 210, 310 may comprise either analyzing the ultrasound scan plane 210, 310 of the anatomy, and automatically positioning the one or more M-mode lines 212, 312, 314 based on the analysis; or positioning each of the one or more M-mode lines 212, 312, 314 at a pre-defined location and orientation in the ultrasound scan plane 210, 310. In certain embodiments, the ultrasound scan plane 210, 310 may be one of an acquired B-mode image or a selected plane in a retrieved three-dimensional (3D) volume.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for assisting the identification of standard ultrasound scan planes based on M-mode analysis.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving and displaying, by at least one processor, an ultrasound scan plane of an anatomy;
positioning, by the at least one processor, one or more M-mode lines in the ultrasound scan plane;
providing, by the at least one processor, an M-mode trace for each of the one or more M-mode lines;
providing, by the at least one processor, feedback based on the M-mode trace for each of the one or more M-mode lines with reference to one or more characteristic trace patterns of a standard view; and
receiving and displaying, by the at least one processor, an updated ultrasound scan plane of the anatomy manipulated in response to the feedback.

2. The method of claim 1, wherein the providing the feedback comprises selecting and displaying one or more reference M-mode trace templates having the one or more characteristic trace patterns of the standard view.

3. The method of claim 1, wherein the providing the feedback comprises:
analyzing, by the at least one processor, the M-mode trace for each of the one or more M-mode lines, and
providing, by the at least one processor, a visual cue, an audio cue, and/or a physical cue indicating whether the standard view has been obtained.

4. The method of claim 1, wherein the providing the feedback comprises:
analyzing, by the at least one processor, the M-mode trace for each of the one or more M-mode lines, and
providing, by the at least one processor, directional feedback to manually manipulate a three-dimensional (3D) volume or an ultrasound probe to obtain the standard view.

5. The method of claim 1, wherein the providing the feedback comprises:
analyzing, by the at least one processor, the M-mode trace for each of the one or more M-mode lines, and
providing, by the at least one processor, directional feedback to automatically manipulate a three-dimensional (3D) volume or an ultrasound acquisition to obtain the standard view.

6. The method of claim 1, wherein the positioning the one or more M-mode lines in the ultrasound scan plane comprises:
analyzing, by the at least one processor, the ultrasound scan plane of the anatomy, and
automatically positioning, by the at least one processor, the one or more M-mode lines based on the analysis.

7. The method of claim 1, wherein the positioning the one or more M-mode lines in the ultrasound scan plane comprises positioning, by the at least one processor, each of the one or more M-mode lines at a pre-defined location and orientation in the ultrasound scan plane.

8. The method of claim 1, wherein the ultrasound scan plane is one of an acquired B-mode image or a selected plane in a retrieved three-dimensional (3D) volume.

9. A system comprising:
at least one processor configured to:
receive an ultrasound scan plane of an anatomy;
position one or more M-mode lines in the ultrasound scan plane;
provide an M-mode trace for each of the one or more M-mode lines;
provide feedback based on the M-mode trace for each of the one or more M-mode lines with reference to one or more characteristic trace patterns of a standard view; and
receive an updated ultrasound scan plane of the anatomy manipulated in response to the feedback; and
a display system configured to present the ultrasound scan plane, the one or more M-mode lines, the M-mode trace for each of the one or more M-mode lines, and the updated ultrasound scan plane.

10. The system of claim 9, wherein the at least one processor is configured to provide the feedback by selecting and displaying one or more reference M-mode trace templates having the one or more characteristic trace patterns of the standard view.

11. The system of claim 9, wherein the at least one processor is configured to provide the feedback by:
analyzing the M-mode trace for each of the one or more M-mode lines, and
providing a visual cue, an audio cue, and/or a physical cue indicating whether the standard view has been obtained.

12. The system of claim 9, wherein the at least one processor is configured to provide the feedback by:
analyzing the M-mode trace for each of the one or more M-mode lines, and
providing directional feedback to manually manipulate a three-dimensional (3D) volume or an ultrasound probe to obtain the standard view.

13. The system of claim 9, wherein the at least one processor is configured to provide the feedback by:
analyzing the M-mode trace for each of the one or more M-mode lines, and
providing directional feedback to automatically manipulate a three-dimensional (3D) volume or an ultrasound acquisition to obtain the standard view.

14. The system of claim 9, wherein the at least one processor is configured to position the one or more M-mode lines in the ultrasound scan plane by either:
analyzing the ultrasound scan plane of the anatomy, and
automatically positioning the one or more M-mode lines based on the analysis; or
positioning each of the one or more M-mode lines at a pre-defined location and orientation in the ultrasound scan plane.

15. The system of claim 9, wherein the ultrasound scan plane is one of an acquired B-mode image or a selected plane in a retrieved three-dimensional (3D) volume.

16. A non-transitory computer readable medium having stored thereon, a computer program having at least one code section, the at least one code section being executable by a machine for causing the machine to perform steps comprising:
receiving and displaying an ultrasound scan plane of an anatomy;
positioning one or more M-mode lines in the ultrasound scan plane;
providing an M-mode trace for each of the one or more M-mode lines;
providing feedback based on the M-mode trace for each of the one or more M-mode lines with reference to one or more characteristic trace patterns of a standard view; and
receiving and displaying an updated ultrasound scan plane of the anatomy manipulated in response to the feedback.

17. The non-transitory computer readable medium of claim 16, wherein the providing the feedback comprises selecting and displaying one or more reference M-mode trace templates having the one or more characteristic trace patterns of the standard view.

18. The non-transitory computer readable medium of claim 16, wherein the providing the feedback comprises:
analyzing the M-mode trace for each of the one or more M-mode lines; and
one of:
providing a visual cue, an audio cue, and/or a physical cue indicating whether the standard view has been obtained;
providing directional feedback to manually manipulate a three-dimensional (3D) volume or an ultrasound probe to obtain the standard view; or
providing directional feedback to automatically manipulate a three-dimensional (3D) volume or an ultrasound acquisition to obtain the standard view.

19. The non-transitory computer readable medium of claim 16, wherein the positioning the one or more M-mode lines in the ultrasound scan plane comprises either:
analyzing the ultrasound scan plane of the anatomy, and
automatically positioning the one or more M-mode lines based on the analysis; or
positioning each of the one or more M-mode lines at a pre-defined location and orientation in the ultrasound scan plane.

20. The non-transitory computer readable medium of claim 16, wherein the ultrasound scan plane is one of an acquired B-mode image or a selected plane in a retrieved three-dimensional (3D) volume.
